(19) United States
(12) Patent Application Publication
(10) Pub. No.: US 2007/0105939 A1
(43) Pub. Date: May 10, 2007

(54) Mesylate salt of 5-(2-dimethylaminoethoxy)-1H-indole-2-carboxylic acid [3-(4-hydroxycarbamoylphenyl)prop-2-ynyl]amide

(75) Inventor: Erik J. Verner, San Mateo, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Pharmacyclics, Inc., Sunnyvale, CA (US)

(21) Appl. No.: 11/441,383

(22) Filed: May 24, 2006

Related U.S. Application Data
(60) Provisional application No. 60/684,492, filed on May 25, 2005.

(51) Int. Cl. A61K 31/405; C07D 209/42

(52) U.S. Cl. 514/419; 548/492

(57) ABSTRACT

The present invention is directed to the mesylate salt of 5-(2-dimethylamino-ethoxy)-1H-indole-2-carboxylic acid [3-(4-hydroxycarbamoylphenyl)prop-2-ynyl]amide, pharmaceutical compositions and processes for preparing the same.

### CROSS-REFERENCES TO RELATED APPLICATIONS

This application claims benefit of Provisional Patent Application Ser. No. 60/684,492, filed May 25, 2005, the disclosure of which is incorporated herein by reference.

### STATEMENT AS TO RIGHTS TO INVENTIONS MADE UNDER FEDERALLY SPONSORED RESEARCH OR DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK

Not Appplicable

### BACKGROUND OF THE INVENTION

1. Field of the Invention

The present invention is directed to the mesylate salt of 5-(2-dimethylamino-ethoxy)-1H-indole-2-carboxylic acid [3-(4-hydroxycarbamoylphenyl)prop-2-ynyl]amide, pharmaceutical compositions and processes for preparing the same.

2. State of the Art

PCT application publication No. WO 2004/019174 specifically discloses 5-(2-dimethyl-aminoethoxy)-1H-indole-2-carboxylic acid [3-(4-hydroxycarbamoyl-phenyl)prop-2-ynyl]amide and its hydrochloride salt as inhibitors of histone deacetylase enzyme H1 and therefore useful in the treatment of a disease mediated by histone deacetylase enzyme H1 such as cancer. 5-(2-Dimethylaminoethoxy)-1H-indole-2-carboxylic acid [3-(4-hydroxycarbamoyl-phenyl)prop-2-ynyl]amide has very good safety and efficacy profile therefore making it attractive for development as a drug.

Unfortunately, 5-(2-dimethylaminoethoxy)-1H-indole-2-carboxylic acid [3-(4-hydroxy-carbamoylphenyl)prop-2-ynyl]amide and its hydrochloride salt suffer from a number of drawbacks. First, they have poor oral bioavailability and have to be administered parenterally in order to have therapeutic effect. Second, they have poor solubility and third, 5-(2-dimethylaminoethoxy)-1H-indole-2-carboxylic acid [3-(4-hydroxy-carbamoylphenyl)prop-2-ynyl]amide hydrochloride is an amorphous solid. Accordingly, efforts were undertaken to identify a new salt form of 5-(2-dimethyl-aminoethoxy)-1H-indole-2-carboxylic acid [3-(4-hydroxy-carbamoylphenyl)prop-2-ynyl]amide that would have greater solubility and/or be crystalline in nature.

A crystalline form of a drug compound is desirable since it has advantages over an amorphous form in several respects. For example, the compound can be easily purified by crystallisation. Furthermore, a crystalline form is usually more stable than an amorphous form, both before and during formulation and during subsequent storage.

It is known that there is no generally applicable method for preparing a crystalline form of an amorphous material. Indeed, it is impossible to know, whether any crystalline form of a given compound exists. In general, extensive experimentation is usually done before a process is identified from which the crystalline form can be isolated. The correct combination of several independently variable conditions (for example, solvent concentration, solvent composition, temperature, cooling rate) must be identified empirically through trial and error with no guarantee of success.

It has now been surprisingly found that the mesylate salt of 5-(2-dimethylaminoethoxy)-1H-indole-2-carboxylic acid [3-(4-hydroxycarbamoyl-phenyl)prop-2-ynyl]amide exists in both the amorphous and crystalline forms thereby making it possible to avoid the above mentioned drawbacks associated with an amorphous drug substance.

### SUMMARY OF THE INVENTION

In a first aspect, this invention is directed to the mesylate salt of 5-(2-dimethylamino-ethoxy)-1H-indole-2-carboxylic acid [3-(4-hydroxycarbamoylphenyl)prop-2-ynyl]amide having the formula shown below and hereinafter referred to as a compound of Formula (I):

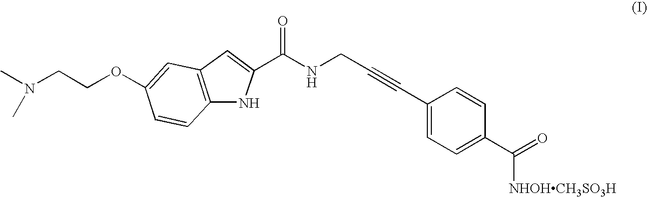

In a second aspect, this invention is directed to an amorphous form of the compound of Formula (I).

In a third aspect, this invention is directed to a crystalline form of the compound of Formula (I).

In a fourth aspect, this invention is directed to a pharmaceutical composition comprising a therapeutically effective amount of a compound of Formula (I) and a pharmaceutically acceptable excipient.

In a fifth aspect, this invention is directed to a method for treating a disease in an animal which is mediated by HDAC which method comprises administering to the animal a pharmaceutical composition comprising a therapeutically effective amount of a compound of Formula (I) and a pharmaceutically acceptable excipient. Preferably, the disease is a proliferative disorder such as cancer and bipolar disorders and the animal is human. Preferably, the cancer is prostate cancer, breast cancer, lung melanoma, stomach cancer, neuroblastoma, colon cancer, pancreatic cancer, ovarian cancer, AML, MML, CML, and T-cell lymphoma.

In a sixth aspect, this invention is directed to a method for treating cancer in an animal which method comprises administering to the animal a pharmaceutical composition comprising a therapeutically effective amount of a compound of Formula (I) and a pharmaceutically acceptable excipient in combination with radiation therapy and optionally in combination with one or more compound(s) independently selected from an estrogen receptor modulator, an androgen receptor modulator, retinoid receptor modulator, a cytotoxic agent, another antiproliferative agent, a prenyl-protein transferase inhibitor, an HMG-CoA reductase inhibitor, an HIV protease inhibitor, a reverse transcriptase inhibitor, or an angiogenesis inhibitor.

In a seventh aspect, this invention is directed to a method for treating cancer in an animal which method comprises administering to the animal a pharmaceutical composition comprising a therapeutically effective amount of a compound of a compound of Formula (I) and a pharmaceutically acceptable excipient in combination with one or more compound(s) independently selected from an estrogen receptor modulator, an androgen receptor modulator, a retinoid receptor modulator, a cytotoxic agent, another antiproliferative agent, a prenyl-protein transferase inhibitor, an HMG-CoA reductase inhibitor, an HIV protease inhibitor, a reverse transcriptase inhibitor, or an angiogenesis inhibitor.

In an eighth aspect, this invention is directed to the use of a compound of Formula (I) for the manufacture of a medicament. Preferably, the medicament is useful in the treatment of a disease mediated by HDAC. More preferably, the disease is cancer.

In a ninth aspect, this invention is directed to a process of preparing a crystalline form of the compound of Formula (I) comprising crystallizing it from a mixture of water and organic alcohol except methanol. Preferably, the alcohol is ethanol, n-propanol, 2-propanol, butanol, and the like. Preferably 2-propanol.

### BRIEF DESCRIPTION OF THE DRAWINGS

Not Applicable

### DETAILED DESCRIPTION OF THE INVENTION

Definitions:

Unless otherwise stated, the following terms used in the specification and claims are defined for the purposes of this Application and have the following meanings:

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Treating” or “treatment” of a disease includes:

(1) preventing the disease, i.e. causing the clinical symptoms of the disease not to develop in a mammal that may be exposed to or predisposed to the disease but does not yet experience or display symptoms of the disease;

(2) inhibiting the disease, i.e., arresting or reducing the development of the disease or its clinical symptoms; or

(3) relieving the disease, i.e., causing regression of the disease or its clinical symptoms.

The term “treating cancer” or “treatment of cancer” refers to administration to a mammal afflicted with a cancerous condition and refers to not only to an effect that alleviates the cancerous condition by killing the cancerous cells, but also to an effect that results in the inhibition of growth and/or metastasis of the cancer.

A “therapeutically effective amount” means the amount of a compound of Formula (I) that, when administered to a mammal for treating a disease, is sufficient to effect such treatment for the disease. The “therapeutically effective amount” will vary depending on the compound, the disease and its severity and the age, weight, etc., of the mammal to be treated.

### Utility

The compounds of this invention are inhibitors of histone deacetylase enzymes and are therefore useful in the treatment of proliferative diseases such as cancer such as lung, colon, skin, breast, ovarian, prostate, liver, brain and skin, psoriasis, fibroproliferative disorder such as liver fibrosis, smooth muscle proliferative disorder such as atherosclerosis and restenosis, inflammatory diseases such as arthritis, diseases involving angiogenesis such as diabetic retinopathy, haematopoietic disorder such as anaemia, fungal, parasitic and bacterial infections, viral infection, autoimmune diseases such as arthritis, multiple sclerosis, lupus, allergies, asthma, allergic rhinitis, and organ transplant, and bipolar disorders.

### Testing

The ability of the compounds of this invention to inhibit histone deacetylase enzymes can be tested in vitro and in vivo assays described in biological assays Example 1 and 2 below.

### Administration and Pharmaceutical Compositions

The compound of Formula (I) will be administered systemically, preferably parenterally, in a therapeutically effective amount. The actual amount of the compound of Formula (I), i.e., the active ingredient, will depend upon numerous factors such as the severity of the disease to be treated, the age and relative health of the subject, and other factors such as combination with other therapeutic agents.

Therapeutically effective amounts of compounds of Formula (I) may range from approximately 0.1-50 mg, preferably 0.1-2.5 mg per kilogram body weight of the recipient per day. Thus, for administration to a 70 kg person, the dosage range would most preferably be about 7 mg to 3.50 g per day, preferably 7 mg to 1.75 g per day.

The compound of Formula (I) will be administered as a pharmaceutical composition using a convenient daily dosage regimen, which can be adjusted according to the degree of affliction. The pharmaceutical compositions are comprised of in general, a compound of Formula (I) in combination with at least one pharmaceutically acceptable excipient. Acceptable excipients are non-toxic, aid administration, and do not adversely affect the therapeutic benefit of the compound of Formula (I). Such excipient may be any solid, liquid, or semi-solid that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of Formula (1) based on the total formulation, with the balance being one or more suitable pharmaceutical excipients. Preferably, the compound is present at a level of about 1-80 wt %. Representative pharmaceutical formulations containing a compound of Formula (I) are described below.

As stated previously, the compound of Formula (I) can be administered in combination with known anti-cancer agents. Such known anti-cancer agents include the following: estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors. The compound of Formula (I) are particularly useful when adminsitered in combination with radiation therapy. Preferred angiogenesis inhibitors are selected from the group consisting of a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, and an antibody to VEGFR and EFGR.

“Estrogen receptor modulators” refers to compounds that interfere or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646. Preferred estrogen receptor modulators are tamoxifen and raloxifene.

“Androgen receptor modulators” refers to compounds that interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds that interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide.

“Cytotoxic agents” refer to compounds which cause cell death primarily by interfering directly with the cell's functioning or inhibit or interfere with cell mitosis, including alkylating agents, tumor necrosis factors, intercalators, microtubulin inhibitors, and topoisomerase inhibitors.

Examples of cytotoxic agents include, but are not limited to, tirapazimine, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine) platinum, benzylguanine, glufosfamide, GPX100, (trans, trans, trans)-bis-mu-(hexane-1,6-diamine)-mu-[diamine-platinum(II)]bis[diamine-(chloro)platinum(II)]-tetrachloride, diarizidinylspermine, arsenic trioxide, 1-(11-dodecyl-amino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxy-carminomycin, annamycin, galarubicin, elinafide, MEN10755, and 4-demethoxy-3-deamino-3-aziridinyl-4-methylsulphonyl-daunorubicin (see WO 00/50032).

Examples of microtubulin inhibitors include paclitaxel, docetaxel (also known as Taxotere®, epothilone A, epothilone B, desoxyepothilone A, desoxyepothilone B or their derivatives); vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1 H,12H-benzo[de]pyrano[3′,4′:b,7]-indolizino[1,2b]quinoline-10,13(9H,15H)dione, lurtotecan, 7-[2-(N-isopropylamino)-ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, 6,9-bis[(2-aminoethyl)-amino]benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2, 1-c]quinolin-7-one, and dimesna.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-mannoheptopyranosyl]-adenine, aplidin, ecteinascidin-743, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, leucovorin, alanosine, 11-acetyl-8-(carbamoyloxymethyl)-4-formyl-6-methoxy-14-oxa-1,11-diazatetra cyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabinofuranosyl cytosine, and 3-aminopyridine-2-carboxaldehyde thiosemicarbazone. “Antiproliferative agents” also includes monoclonal antibodies to growth factors, other than those listed under “angiogenesis inhibitors”, such as trastuzumab, and tumor suppressor genes, such as p53, which can be delivered via recombinant virus-mediated gene transfer (see U.S. Pat. No. 6,069,134).

“HMG-CoA reductase inhibitors” refers to inhibitors of 3-hydroxy-3-methylglutaryl-CoA reductase. Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of HMG-CoA reductase” have the same meaning when used herein. It has been reported that (Int. J. Cancer, 20, 97(6):746-50, (2002)) combination therapy with lovastatin, a HMG-CoA reductase inhibitor, and butyrate, an inducer of apoptosis in the Lewis lung carcinoma model in mice showed potentiating antitumor effects

Examples of HMG-CoA reductase inhibitors that may be used include but are not limited to lovastatin (MEVACOR®; see U.S. Pat. Nos. 4,231,938, 4,294,926, and 4,319,039), simvastatin (ZOCOR®; see U.S. Pat. Nos. 4,444,784, 4,820,850, and 4,916,239), pravastatin (PRAVACHOL®; see U.S. Pat. Nos. 4,346,227, 4,537,859, 4,410,629, 5,030,447, and 5,180,589), fluvastatin (LESCOL®; see U.S. Pat. Nos. 5,354,772, 4,911,165, 4,929,437, 5,189,164, 5,118,853, 5,290,946, and 5,356,896), atorvastatin (LIPITOR®; see U.S. Pat. Nos. 5,273,995, 4,681,893, 5,489,691, and 5,342,952) and cerivastatin (also known as rivastatin and BAYCHOL®; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89, Feb. 5, 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and colchicin the use of such salts, esters, open-acid and lactone forms is included within the scope of this invention.

In HMG-CoA reductase inhibitors where an open-acid form can exist, salt and ester forms may preferably be formed from the open-acid, and all such forms are included within the meaning of the term “HMG-CoA reductase inhibitor” as used herein. Preferably, the HMG-CoA reductase inhibitor is selected from lovastatin and simvastatin, and most preferably simvastatin.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase). Examples of prenyl-protein transferase inhibiting compounds include (±)-6-[amino(4-chlorophenyl)(1-methyl-1H-imidazol-5-yl)methyl]-4-(3-chlorophenyl)-1-methyl-2(1H)-quinolinone, (−)-6-[amino(4-chlorophenyl)(1-methyl-1H-imidazol-5-yl)methyl]-4-(3-chloro phenyl)-1-methyl-2(1H)-quinolinone, (+)-6-[amino(4-chlorophenyl)( 1-methyl-1H-imidazol-5-yl)methyl]-4-(3-chloro phenyl)-1-methyl-2(1H)-quinolinone, 5(S)-n-butyl-1-(2,3-dimethylphenyl)-4-[1-(4-cyanobenzyl)-5-imidazolylmethyl]-2-piperazinone, (S)-1-(3-chlorophenyl)-4-[1-(4-cyanobenzyl)-5-imidazolylmethyl]-5-[2-(ethanesulfonyl)-methyl)-2-piperazinone, 5(S)-n-butyl-1-(2-methylphenyl)-4-[1-(4-cyanobenzyl)-5-imidazolylmethyl]-2-piperazinone, 1-(3-chlorophenyl)-4-[1-(4-cyanobenzyl)-2-methyl-5-imidazolylmethyl]-2-piperazinone, 1-(2,2-diphenylethyl)-3-[N-(1-(4-cyanobenzyl)-1H-imidazol-5-ylethyl)carbamoyl]piperidine, 4-{5-[4-hydroxymethyl-4-(4-chloropyridin-2-ylmethyl)-piperidine-1-ylmethyl]-2-methylimidazol-1-ylmethyl}benzonitrile, 4-{5-[4-hydroxy-methyl-4-(3-chlorobenzyl)-piperidine-1-ylmethyl]-2-methylimidazol-1-ylmethyl}-benzonitrile, 4-{3-[4-(2-oxo-2H-pyridin-1-yl)benzyl]-3H-imidazol-4-ylmethyl}-benzonitrile, 4-{3-[4-(5-chloro-2-oxo-2H-[1,2′]bipyridin-5′-ylmethyl]-3H-imidazol-4-ylmethyl}benzonitrile, 4-{3-[4-(2-oxo-2H-[1,2′]bipyridin-5′-ylmethyl]-3H-imidazol-4-ylmethyl}benzonitrile, 4-[3-(2-oxo-1-phenyl-1,2-dihydropyridin-4-ylmethyl)-3H-imidazol-4-ylmethyl}benzonitrile, 18,19-dihydro-19-oxo-5H,17H-6,10: 12,16-dimetheno-1H-imidazo[4,3-c][1,11,4]dioxa-azacyclononadecine-9-carbonitrile, (±)-19,20-dihydro-19-oxo-5H-18,21-ethano-12,14-etheno-6,10-metheno-22H-benzo[d]imidazo[4,3-k][1,6,9,12]-oxatriaza-cyclooctadecine-9-carbonitrile, 19,20-dihydro-19-oxo-5H,17H-18,21-ethano-6,10: 12,16-dimetheno-22H-imidazo[3,4-h][1,8,11,14]oxatriazacyclo-eicosine-9-carbonitrile, and (±)-19,20-dihydro-3-methyl-19-oxo-5H-18,21-ethano-12,14-etheno-6,10-metheno-22H-benzo[d]imidazo[4,3-k][1,6,9,12]oxa-triazacyclooctadecine-9-carbonitrile.

Other examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. Nos. 5,420,245, 5,523,430, 5,532,359, 5,510,510, 5,589,485, 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see J. of Cancer, Vol. 35, No. 9, pp. 1394-1401 (1999).

Examples of HIV protease inhibitors include amprenavir, abacavir, CGP-73547, CGP-61755, DMP-450, indinavir, nelfinavir, tipranavir, ritonavir, saquinavir, ABT-378, AG 1776, and BMS-232, 632. Examples of reverse transcriptase inhibitors include delaviridine, efavirenz, GS-840, HB Y097, lamivudine, nevirapine, AZT, 3TC, ddC, and ddI. It has been reported ((Nat. Med. 8(3):225-32, (2002)) that HIV protease inhibitors, such as indinavir or saquinavir, have potent anti-angiogenic activities and promote regression of Kaposi sarcoma

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR20), inhibitors of epidermal-derived, fibroblast-derived, or platelet derived growth factors, MMP (matrix metalloprotease) inhibitors, integrin blockers, interferon-∝, interleukin-12, pentosan polysulfate, cyclooxygenase inhibitors, including nonsteroidal anti-inflammatories (NSAIDs) like aspirin and ibuprofen as well as selective cyclooxygenase-2 inhibitors like celecoxib, valdecoxib, and rofecoxib (PNAS, Vol. 89, p. 7384 (1992); JNCI. Vol. 69, p. 475 (1982); Arch. Opthalmol. Vol. 108, p. 573 (1990); Anat. Rec. Vol. 238, p. 68 (1994); FEBS Letters, Vol. 372, p. 83 (1995); Clin., Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186). P As described above, the combinations with NSAID's are directed to the use of NSAID's which are potent COX-2 inhibiting agents. Such compounds include, but are not limited to those disclosed in, U.S. Pat. Nos. 5,474,995, 5,861,419, 6,001,843, 6,020,343, 5,409,944, 5,436,265, 5,536,752, 5,550,142, 5,604,260, 5,698,584, 5,710,140, 5,344,991, 5,134,142, 5,380,738, 5,393,790, 5,466,823, 5,633,272, 6,313,138, and 5,932,598, and WO 94/15932, all of which are hereby incorporated by reference. Other examples of specific inhibitors of COX-2 include those disclosed in U.S. Patent the disclosure of which is incorporated herein by reference in its entirety.

General and specific synthetic procedures for the preparation of the COX-2 inhibitor compounds described above are found in U.S. Pat. Nos. 5,474,995, 5,861,419, and 6,001,843, all of which are herein incorporated by reference. Compounds which are specific inhibitors of COX-2 and are therefore useful in the present invention, and methods of synthesis thereof, can be found in the following patents, pending applications and publications, which are herein incorporated by reference: U.S. Pat. Nos. 5,474,995, 5,861,419, 6,001,843, 6,020,343, 5,409,944, 5,436,265, 5,536,752, 5,550,142, 5,604,260, 5,698,584, and 5,710,140.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, 5-amino-1-[[3,5-dichloro-4-(4-chloro-benzoyl)phenyl]-methyl]-1H-1,2,3-triazole-4-carboxamide, CM101, squalamine, combretastatin, RPI4610, NX31838, sulfated mannopentose phosphate, 7,7-(carbonyl-bis[imino-N-methyl-4,2-pyrrolocarbonyl-imino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counter-act binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆; αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some specific examples of tyrosine kinase inhibitors include N-(trifluoromethyl-phenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, ST1571, CEP2563, 4-(3-chlorophenylamino)-5,6-dimethyl-7H-pyrrolo [2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, SU11248, STI571A, N-4-chlorophenyl-4-(4-pyridylmethyl)-1-phthalazinamine, and EMD121974.

The instant compound is also useful in combination with platelet fibrinogen receptor (GP IIb/IIIa) antagonists, such as tirofiban, to inhibit metastasis of cancerous cells. Tumor cells can activate platelets largely via thrombin generation. This activation is associated with the release of VEGF. The release of VEGF enhances metastasis by increasing extravasation at points of adhesion to vascular endothelium (Amirkhosravi, Platelets 10, 285-292, (1999)). Therefore, the present compound can serve to inhibit metastasis in combination with GP IIb/IIIa antagonists. Examples of other fibrinogen receptor antagonists include abciximab, eptifibatide, sibrafiban, lamifiban, lotrafiban, cromofiban, and CT50352.

The compound of this invention can be used with antineoplastic agents such as doxorubicin, carminomycin, daunorubicin, aminopterin, methotrexate, methopterin, dichloromethotrexate, mitomycin C, porfiromycin, Herceptin®, Rituxan®, Avastin®, Tarceva®, 5-fluorouracil, 6-mercaptopurine, gemcitabine, cytosine arabinoside, podophyllotoxin or podophyllotoxin derivatives such as colchicines, etoposide, etoposide phosphate or teniposide, melphalan, vinblastine, vincristine, leurosidine, vindesine, leurosine, estramustine, cisplatin, cyclophosphamide, bleomycin, tamoxifen, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons and interleukins.

If formulated as a fixed dose, such combination products employ the compound of this invention within the dosage range described above and the other pharmaceutically active agent(s) within its approved dosage range. Compound of the instant invention may alternatively be used sequentially with known pharmaceutically acceptable agent(s) when a combination formulation is inappropriate.

The term administration (e.g., “administering” a compound) in reference to the compound of the invention means introducing the compound into the system of the animal in need of treatment. When the compound of the invention is provided in combination with one or more other active agents (e.g., a cytotoxic agent, etc.), “administration” is understood to include concurrent and sequential introduction of the compound and other agents.

As used herein, the term “composition” is intended to encompass a product comprising the specified ingredients in the specified amounts, as well as any product which results, directly or indirectly, from combination of the specified ingredients in the specified amounts.

Radiation therapy, including x-rays or gamma rays that are delivered from either an externally applied beam or by implantation of tiny radioactive sources, may also be used in combination with the compounds of this invention alone to treat cancer.

### EXAMPLES

### Synthetic Example

General Comments:

Except where noted below, all solvents and reagents were purchased from Aldrich and used as received. All reactions and products were analyzed for chemical purity using reverse-phase HPLC and are reported in area percent relative to other components that were detected at the wavelength used for the analysis. In-process monitoring of reactions was performed using an Agilent HP1100 HPLC, fitted with Diode Array Detector and a Polaris 5μ 50×2.0 mm column [Varian part number A2000-050X020] maintained at 40.0° C. Samples were analyzed using a gradient elution from 2% to 95% organic buffer over a 5-min period, at a flow rate of 1 mL/min. The organic mobile phase consisted of acetonitrile containing 0.05% trifluoroacetic acid (TFA) and the aqueous mobile phase consisted of water containing 0.05% TFA. All purity data reported for HPLC analyses are given as area percent purities. Filtration or purification of crude products through silica gel was performed with 230-400 mesh SiO₂ (EMD part number 9385-9). Thin-Layer chromatography was performed using Silica Gel 60 F₂₅₄ (EMD part number 15341-10) with the solvent system described in the pertinent section of the experimental. Combustion analyses were performed by Robertson Microlit Laboratories, Inc., Madison, N.J. Proton- and Carbon-NMR spectra were obtained at 400 MHz and 100 MHz respectively using a Varian Mercury instrument and Gemini processing software.

### Example 1

### Synthesis of mesylate salt of 5-(2-dimethylaminoethoxy)-1H-indole-2-carboxylic acid [3-(4-hydroxycarbamoylphenyl)prop-2-ynyl]amide

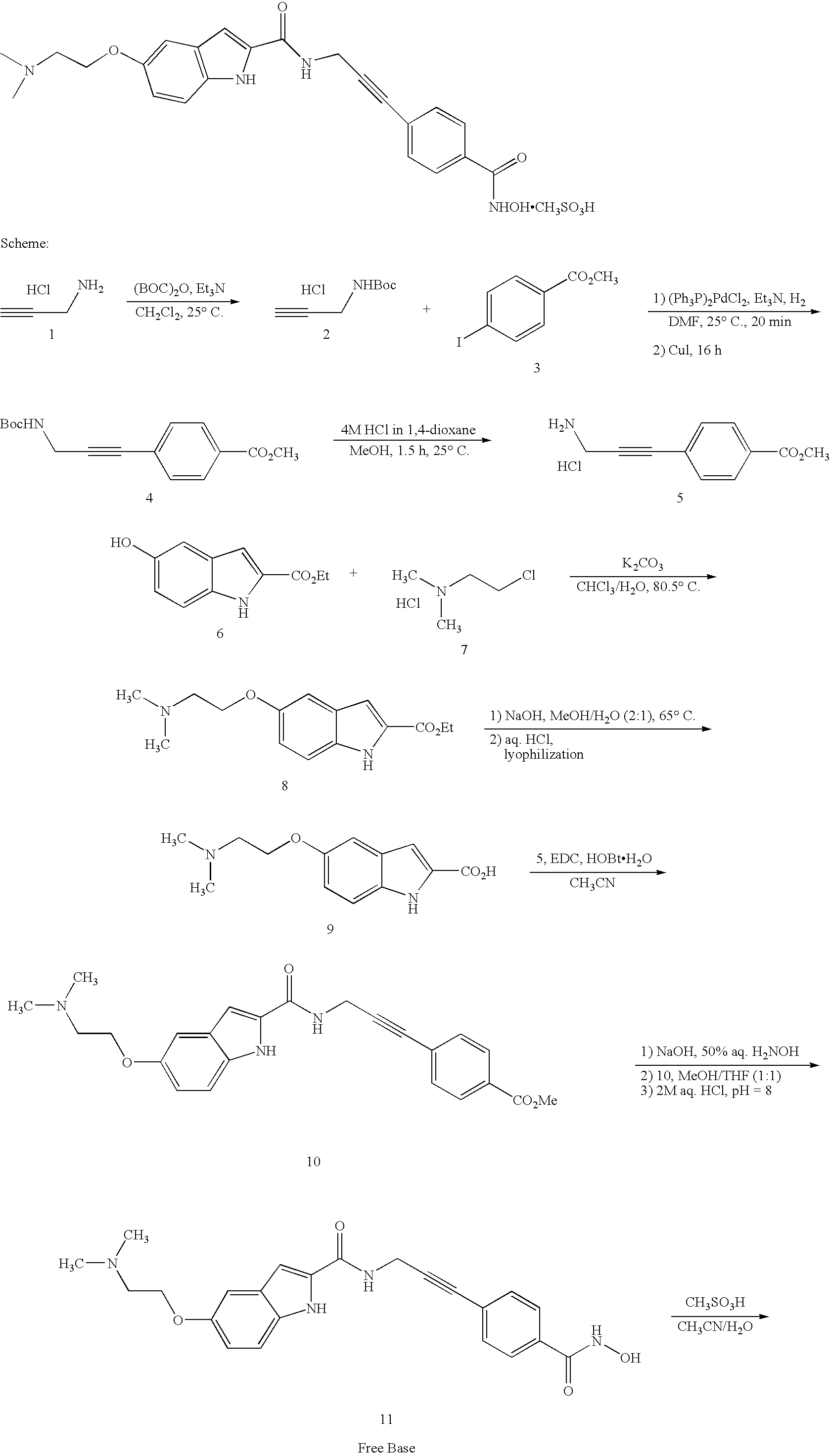 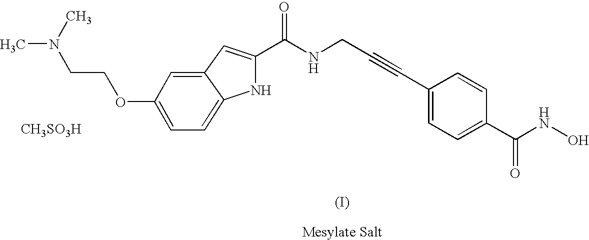 Step 1

To a suspension of propargylamine hydrochloride (25 g, 273.1 mmol) in anhydrous THF (250 mL) were added in sequence Et₃N (38.06 mL, 273.1 mmol), di-tert-butyl dicarbonate (59.6 g, 273.1 mmol), and Et₃N (38.06 mL, 273.1 mmol). After stirring at room temperature for 15 min, additional Et₃N (19.03 mL, 136.6 mmol) and water (˜20 mL) were added and gas evolution was monitored. After stirring for 30 min, no further gas evolution was observed and TLC [EtOAc/hexane, (1:2)] showed complete consumption of propargylamine hydrochloride. The reaction was quenched with 0.25M aqueous HCl (˜300 mL) until the solution became clear and diluted with EtOAc (250 mL). The organic phase was separated and washed with 0.5M aqueous HCl, brine, then dried over sodium sulfate. Concentration in vacuo provided crude 1-N-(tert-butyloxycarbonylamino)-2-propyne (40.35 g, 95.2%) which became a white solid after drying under high vacuum overnight. The crude product was used in the next step without further purification.

Step 2

To a suspension of 1-N-(tert-butyloxycarbonylamino)-2-propyne (40.30 g, 259.7 mmol) and methyl 4-iodobenzoate (68.05 g, 259.7 mmol) in DMF (EMD, 200 mL) was added triethylamine (163 mL, 1.17 mol). The greenish solution was evacuated and purged with nitrogen. Nitrogen was bubbled through the stirred reaction mixture for an additional 20 min. trans-Dichlorobis(triphenylphosphine)palladium(II) (Strem part number 46-0530, Lot # B5876121, 1.83 g, 2.6 mmol) was added in one portion and the nitrogen line was replaced by a septum with a balloon filled with hydrogen. After stirring for 25 min, CuI (990 mg, 5.2 mmol) was added in one portion. After stirring for 16 h, the reaction mixture was diluted with brine and EtOAc. The separated aqueous layer was extracted with EtOAc and the combined organic layers were washed with 0.5M aqueous HCl, saturated aqueous NH₄Cl, brine, and dried over sodium sulfate. After removal of the solvent in vacuo, the dark residue was dissolved in diethyl ether (˜300 mL) and filtered through a small pad of silica gel and eluted with diethyl ether/hexane (4:1) to remove polar impurities and residual catalyst. Concentration in vacuo provided crude 4-(3-tert-butoxycarbonylamino-prop-1-ynyl)-benzoic acid methyl ester (76.16 g) as brownish solid. This material was used in the next step without further purification.

Step 3

To a suspension of 4-(3-tert-butoxycarbonylaminoprop-1-ynyl)benzoic acid methyl ester (76.0 g, 337 mmol) in methanol (150 mL) was added 4M HCl in 1,4-dioxane (150 mL, 600 mmol) slowly in several portions over a period of 90 min at ambient temperature while monitoring the resulting gas evolution. After stirring for an additional 30 min, the volume of solvent was reduced by ˜150 mL by evaporation in vacuo to remove the methanol. Diethyl ether (˜700 mL) was added with vigorous stirring. The resulting precipitate was filtered, washed with diethyl ether, and dried in vacuo to provide 4-(3-amino-prop-1-ynyl)benzoic acid methyl ester hydrochloride (50.4 g) as a grayish, shiny solid. This material was used in the next step without further purification.

Step 4

To a suspension of 2-hydroxy-1H-indole-2-carboxylic acid ethyl ester (10.08 g, 49.11 mmol) in CHCl₃ (200 mL) was added 2-(dimethylamino)ethyl chloride hydrochloride (10.57 g, 73.37 mmol,), potassium carbonate (33.88 g, 245.2 mmol), and water (40 mL) in sequence. The stirred solution was placed in a 65° C. oil bath and the temperature was slowly raised to 80.5° C. over 65 min. After 7 h 40 min, the reaction mixture was cooled to room temperature and the phases were separated. The organic phase was reduced in vacuo to 25% of its original volume. The residue was combined with the aqueous phase and diluted with water and toluene. The organic phase was separated and washed with water and extracted with 1M aqueous HCl. The acidic aqueous phase was washed with toluene, basified by the addition of ˜4M aqueous NaOH (55 mL), and extracted with toluene. The organic extract was washed with water, brine, dried over sodium sulfate, and concentrated in vacuo to provide 5-[(2-dimethylamino)ethoxy]-1H-indole-2-carboxylic acid ethyl ester (12.06 g, 88.9%) as an off-white solid.

Step 5

A solution of 5-[(2-dimethylamino)ethoxy]-1H-indole-2-carboxylic acid ethyl ester (16.35 g, 59.18 mmol) in methanol (225 mL) was treated with a solution of NaOH (2.74 g) in 75 mL water and then heated at 80° C. (external oil bath temperature). After 50 min, the reaction mixture was cooled to room temperature and concentrated HCl was added to bring the pH of the solution to <3. The reaction mixture was concentrated in vacuo and the residue was treated with water to prevent crystallization and then the solution was lyophilized to afford crude 5-[(2-dimethylamino)ethoxy]-1H-indole-2-carboxylic acid (21.29 g), which was contaminated with NaCl. A portion of crude 5-[(2-dimethylamino)ethoxy]-1H-indole-2-carboxylic acid (4.408 g, 12.25 mmol; based on a potency of 69% due to the added weight of NaCl) was suspended in water (22 mL) and treated with 4N aqueous NaOH (˜4 mL, ˜16 mmol) to affect dissolution. The solution was filtered through Celite® to remove traces of fine particulate matter and the clarified filtrate was treated with concentrated HCl to adjust the pH of the clarified basic solution from pH=10.0 to pH=6.54. The pH adjustment caused precipitation of the product as an amorphous solid. The suspension was heated to affect complete dissolution then the solution was gradually cooled to ambient temperature. The resulting solid was filtered, washed with a minimum of cold water and then acetone to afford 5-[(2-dimethylamino)ethoxy]-1H-indole-2-carboxylic acid (2.89 g, 95.1%) as a white, crystalline material in a purity of >99% as determined by HPLC and ¹H NMR analyses.

Step 6

To a suspension of 5-[(2-dimethylamino)ethoxy]-1H-indole-2-carboxylic acid obtained from the initial lypohilization step (16.031 g) in acetonitrile (900 mL) was added EDC hydrochloride (15.456 g, 80.62 mmol) and HOBt hydrate (8.251 g, 53.87 mmol) in sequence. After 186 min, 4-(3-amino-prop-1-ynyl)benzoic acid methyl ester hydrochloride (10.066 g, 44.598 mmol) and Et₃N (11.3 mL, 81 mmol) were added in sequence. After stirring for 15 h, 30 min, the reaction mixture had thickened. Most of the reaction solvent was removed in vacuo at 35° C. (bath temperature) and the residue was partitioned between EtOAc, water, and saturated aqueous NaHCO₃. The separated aqueous layer was extracted with EtOAc and the combined organic layers were washed with saturated aqueous NaHCO₃, water, brine, and dried over sodium sulfate. The product 4-(3-{5-[(2-dimethylamino)ethoxy]-1H-indole-2-carbonyl]amino}-prop-1-ynyl)-benzoic acid methyl ester began to crystallize during the filtration of the drying agent. The filtrate was combined with the crystalline material dissolved by the addition of EtOAc. Removal of a significant portion of EtOAc in vacuo provided the product as a crystalline material, which was collected by filtration, washed with EtOAc and hexanes and dried. Analysis by TLC indicated it contained basline impurity. The baseline impurity was removed by filtering the impure product through a plug of silica gel as a solution in THF and eluting with THF/methanol (9:1). Concentration in vacuo and re-crystallization from THF/Hexanes (the residue was taken up in THF and diluted with the same volume of hexane) afforded pure product (13.24 g, 71%) as a white solid. Additional material was recovered from the EtOAc and the THF/hexanes liquors. Purification of the residues from the combined liquors by re-chromatography using THF/methanol (19:1) provided an additional 2.31 g (12%) the product.

Step 7

Solid NaOH (7.97 g, 199 mmol) was dissolved in 50% aqueous hydroxylamine (76.3 mL, 1.25 mol) at 10-25° C. (ice bath). A solution of 4-(3-{5-[(2-dimethylamino)ethoxy]-1H-indole-2-carbonyl]amino}prop-1-ynyl)-benzoic acid methyl ester (10.45 g, 24.9 mmol) in THF/methanol (700 mL, 1:1) was added to the vigorously stirred solution of the above aqueous hydroxylamine over a period of ˜10 min. The initial biphasic solution became homogenous during the course of the addition. After stirring for additional 5 min, the reaction mixture was concentrated in vacuo in order to remove the methanol and THF. To the oily, orange, and biphasic residue was added water and the reaction mixture was cooled to 0° C. The pH of the mixture was adjusted to pH ˜8 via the slow addition of 2M aqueous HCl. The resulting precipitate was collected, washed with water, and dried in vacuo to provide 5-[(2-dimethyl-amino)ethoxy]-1H-indole-2-carboxylic acid [3-(4-hydroxycarbamoylphenyl)prop-2-ynyl]amide free base (10.38 g, 97%) as an off-white solid.

Step 8

A vigorously stirred suspension of 5-[(2-dimethylamino)ethoxy]-1H-indole-2-carboxylic acid [3-(4-hydroxycarbamoylphenyl)prop-2-ynyl]amide free base (30.20 g, 71.83 mmol) in acetonitrile (250 mL) was cooled to 0° C. and treated with a solution of methanesulfonic acid (3.367 mL, 51.92 mmol, 100 mole %) in water (˜100 mL). The reaction mixture was allowed to warm up to room temperature and acetonitrile (total of ˜600 mL) and water (total of ˜1.2 L) were added in portions to give a clear, orange solution. The solution was filtered to remove traces of insoluble fine particulate matter. Lyophilization of the resulting filtrate provided the title compound (36.25 g, 97.7%) as an amorphous off white solid.

### Preparation of crystalline 5-(2-dimethylaminoethoxy)-1H-indole-2-carboxylic acid [3-(4-hydroxycarbamoylphenyl)prop-2-ynyl]amide mesylate salt

Amorphous 5-(2-dimethylaminoethoxy)-1H-indole-2-carboxylic acid [3-(4-hydroxy-carbamoylphenyl)prop-2-ynyl]amide mesylate salt (3.88 g, 7.71 mmol) was suspended in water (10 mL) and treated with 2-propanol (40 mL). The reaction mixture was placed in an 80° C. oil bath. Additional water (11.5 mL) was added to give a homogeneous solution at 80° C. 2-Propanol (57 mL) was added until the solution showed the first signs of remaining turbid at 80° C. The sample was sealed and allowed to cool to ambient temperature overnight. The crystallized product was filtered, washed with small amounts of 2-propanol, and ether, and dried (high vacuum) to afford 5-(2-dimethylaminoethoxy)-1H-indole-2-carboxylic acid [3-(4-hydroxycarbamoylphenyl)prop-2-ynyl]amide mesylate salt (2.21 g, 57%) as a crystalline solid, demonstrated by X-ray powder diffraction studies.

### BIOLOGICAL EXAMPLES

### Example 1

### Inhibition of HDAC in Vitro

The HDAC inhibitory activity of the compounds of this invention in vitro was determined as follows.

Measurements were performed in a reaction volume of 100 μL using 96-well assay plates. HDAC-1 (200 pM final concentration) in reaction buffer (50 mM HEPES, 100 mM KCl, 0.001% Tween-20, 5% DMSO, pH 7.4) was mixed with inhibitor at various concentrations and allowed to incubate for 30 min, after which trypsin and acetyl-Gly-Ala-(N-acetyl-Lys)-AMC were added to final concentrations of 50 nM and 25 □M, respectively, to initiate the reaction. Negative control reactions were performed in the absence of inhibitor in replicates of eight.

The reactions were monitored in a fluorescence plate reader. After a 30 min lag time, the fluorescence was measured over a 30 min time frame using an excitation wavelength of 355 nm and a detection wavelength of 460 nm. The increase in fluorescence with time was used as the measure of the reaction rate. Inhibition constants were obtained using the program BatchKi (Kuzmic et al. Anal. Biochem. 286, 45-50, (2000).

### Example 2

### Cell Proliferation Assay in Vitro

The ability of the compounds of Formula (I) to inhibit growth of tumor cells in vitro was determined as follows.

Stock cultures of the DU 145 prostate carcinoma cell line were maintained in RPMI medium 1640 containing 10% (v/v) fetal bovine serum, 2 mM L-glutamine, 1 mM sodium pyruvate, 50 units/ml penicillin, and 50 μg/ml streptomycin at 37° C. in 5% CO₂ humidified atmosphere. Cells were cultured in 75-cm² culture flasks and subcultures were established every 3 to 4 days so as not to allow the cells to exceed 90% confluence.

DU 145 cells were harvested for proliferation assays by trypsinization (0.05% trypsin/0.53 mM EDTA), washed twice in culture medium, resuspended in appropriate volume of medium, and then counted using a hemacytometer. Cells were seeded in wells of flat-bottom 96-well plates at a density of 5,000 cell/well in 100 μl. Cells were allowed to attach for 1.5 to 2 h at 37° C.

Compounds were diluted from 10 mM stock solutions in DMSO. Serial 3-fold dilutions were performed in medium containing 0.6% DMSO in wells (in triplicate) of a 96-well U-bottom plates starting with a 60 μM solution. After dilutions were completed, 100 μl of each compound dilution (in triplicate) was transferred to designated triplicate wells of the 96-well plate containing cells in 100 μl of medium. Final concentrations of the dose-response for compounds in assay plates ranged from 0.12 to 30 μM. Control wells (cells with no treatment) received 100 μl of 0.6% DMSO in culture medium. Wells containing medium with no cells served as the background wells. Cells were cultured with the compounds for 48 and 72 h at 37° C. in a humidified CO₂ incubator.

Cell proliferation was assessed by measuring fluorescence after the addition of the fluorogenic redox indicator, Alamar Blue™ (BioSource International). Ten μl of Alamar Blue™ was added to each well of the 96-well plate(s) 3 to 4 h prior to the end of the incubation period. Assay plates were read in a fluorescence plate reader (excitation, 530 nm; emission, 620 nm). GI₅₀ values (concentration at which the growth of the tumor cells was inbibited by 50%) for compounds were determined by plotting the percent control fluorescence against the logarithm of the compound concentration. The compounds of this invention inhibited the growth of the tumor cells.

### Pharmaceutical Composition Examples

The following are the parenteral pharmaceutical formulations containing 5-(2-dimethylaminoethoxy)-1H-indole-2-carboxylic acid [3-(4-hydroxycarbamoylphenyl)prop-2-ynyl]amide as the HCl and the mesylate salt.

### Injectable Formulations

### Formulation No. 1

The following ingredients are mixed to form an injectable formulation.

| Ingredient | Amount | |
|---|---|---|
| Compound of this invention | 2.57 | mg |
| 5% dextrose in water | 0.95 | mL |
| DMSO | 0.05 | mL |

### Formulation No. 2

The following ingredients are mixed to form an injectable formulation.

| Ingredient | Amount | |
|---|---|---|
| Compound of this invention | 10 | mg |
| Captisol ® (20% in sterile water) | 1 | mL |

### Formulation No. 3

The following ingredients are mixed to form an injectable formulation.

| Ingredient | Amount | |
|---|---|---|
| HCl salt | 5 | mg |
| Trappsol ® (20% in sterile water) | 1 | mL |

### Formulation No. 4

The following ingredients are mixed to form an injectable formulation.

| Ingredient | Amount | |
|---|---|---|
| Compound of the invention | 10 | mg |
| Trappsol ® (20% in sterile water) | 1 | mL |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled. All patents, patent applications and publications cited in this application are hereby incorporated by reference in their entirety for all purposes to the same extent as if each individual patent, patent application or publication were so individually denoted.

What is claimed is:

1. Mesylate salt of 5-(2-dimethylamino-ethoxy)-1H-indole-2-carboxylic acid [3-(4-hydroxycarbamoylphenyl)prop-2-ynyl]amide having the structure:

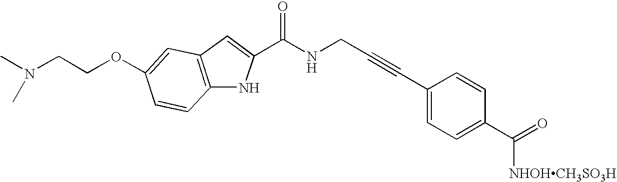

2. The compound of claim 1 having amorphous form.

3. The compound of claim 1 having crystalline form.

4. A pharmaceutical composition comprising a therapeutically effective amount of a compound of any of the claim 1-3 and a pharmaceutically acceptable excipient.

5. A method for treating a disease in an animal which is mediated by HDAC which method comprises administering to the animal a pharmaceutical composition comprising a therapeutically effective amount of a compound of any of the claims 1-3 and a pharmaceutically acceptable excipient.

6. The method of claim 6 wherein the disease is cancer.

7. A method for treating cancer in an animal which method comprises administering to the animal a pharmaceutical composition comprising a therapeutically effective amount of a compound of any of the claim 1-3 and a pharmaceutically acceptable excipient in combination with radiation therapy and optionally in combination with one or more compound(s) independently selected from an estrogen receptor modulator, an androgen receptor modulator, a retinoid receptor modulator, a cytotoxic agent, another antiproliferative agent, a prenyl-protein transferase inhibitor, an HMG-CoA reductase inhibitor, an HIV protease inhibitor, a reverse transcriptase inhibitor, or an angiogenesis inhibitor.

8. A method for treating cancer in an animal which method comprises administering to the animal a pharmaceutical composition comprising a therapeutically effective amount of a compound of any of the claim 1-3 and a pharmaceutically acceptable excipient in combination with one or more compound(s) independently selected from an estrogen receptor modulator, an androgen receptor modulator, a retinoid receptor modulator, a cytotoxic agent, another antiproliferative agent, a prenyl-protein transferase inhibitor, an HMG-CoA reductase inhibitor, an HIV protease inhibitor, a reverse transcriptase inhibitor, or an angiogenesis inhibitor.

9. The use of a compound of any of the claims 1-3 for the manufacture of a medicament.

10. The use according to claim 8 wherein the medicament is useful in the treatment of a disease mediated by HDAC.

11. The use according to claim 9 wherein the disease is cancer.

12. A process for preparing crystalline 5-(2-dimethylamino-ethoxy)-1H-indole-2-carboxylic acid [3-(4-hydroxycarbamoylphenyl)prop-2-ynyl]amide mesylate salt comprising crystallizing 5-(2-dimethylamino-ethoxy)-1H-indole-2-carboxylic acid [3-(4-hydroxycarbamoylphenyl)prop-2-ynyl]amide mesylate salt from a mixture of water and alcohol provided that the alcohol is not methanol.

13. The process of claim 12 wherein the alcohol is 2-propanol.
